(19) United States
(12) Patent Application Publication
(10) Pub. No.: US 2007/0212372 A1
(43) Pub. Date: Sep. 13, 2007

(54) Methods for the treatment and prophylaxis of demeyelinating disease

(75) Inventors: Kylie Ann-Maree Shipham, Donvale (AU); Tamara Ann Michelle Bucci, Brunswick (AU); Helmut Butzkueven, Kensington (AU); Trevor John Kilpatrick, Parkville (AU); Perry Francis Bartlett, (US); Merja Soilu-Hanninen, Turku (FI)

Correspondence Address: Edward W. Grolz;Scully, Scott, Murphy & Presser, 400 Garden City Plaza, Garden City, NY, 11530, US

(73) Assignee: Zenyth Operations Pty Ltd, Richmond (AU)

(21) Appl. No.: 11/799,862

(22) Filed: May 3, 2007

Related U.S. Application Data
(63) Continuation of application No. 10/220,654, filed on Apr. 9, 2003, now abandoned.

(30) Foreign Application Priority Data
Mar. 3, 2000 (AU) PQ 5984
May 29, 2000 (AU) PQ 7810

(51) Int. Cl. A61K 39/00

(52) U.S. Cl. 424/198.1

(57) ABSTRACT

The present invention relates to a method for the treatment and/or prophylaxis of a nervous system disease, demyelinating disease and/or an inflammatory disease of either the central or peripheral nervous system such as but not limited to an encephalopathic condition and even more particularly an encephalomyelopathic condition. The present invention further provides the use of leukemia inhibitory factor or derivatives, homologues or analogues thereof in the treatment and/or prophylaxis of a nervous system disease, demyelinating disease and/or an inflammatory disease of either the central or peripheral nervous system. Leukemia inhibitory factor may be used alone or in combination with one or more other therapeutic molecules.

### FIELD OF THE INVENTION

The present invention relates generally to a method of treatment and to agents useful for same. More particularly, the present invention contemplates a method for the treatment and/or prophylaxis of a nervous system disease, demyelinating disease and/or an inflammatory disease of either the central or peripheral nervous system such as but not limited to an encephalopathic condition and even more particularly an encephalomyelopathic condition. The present invention further provides the use of leukemia inhibitory factor or derivatives, homologues or analogues thereof in the manufacture of a medicament for the treatment and/or prophylaxis of a nervous system disease, demyelinating disease and/or an inflammatory disease of either the central or peripheral nervous system such as but not limited to an encephalopathic and more particularly an encephalomyelopathic condition. Leukemia inhibitory factor may be used alone or in combination with one or more other therapeutic molecules such as but not limited to other cytokines. The method of the present invention enables the development of a therapeutic protocol for conditions such as multiple sclerosis, optic neuritis and other single episodes of central demyelination, transverse myelitis, HEV-induced leucoencephalopathy or chemotherapy induced leucoencephalopathy.

### BACKGROUND OF THE INVENTION

Reference to any prior art in this specification is not, and should not be taken as, an acknowledgement or any form of suggestion that this prior art forms part of the common general knowledge in Australia or any other country.

Bibliographic details of the publications numerically referred to in this specification are collected at the end of the description.

Leukemia inhibitory factor (LIF) is a cytokine which exhibits a range of activities on a number of different cell types. For example, LIF is capable of maintaining embryonic stem (ES) cells in culture while retaining pluripotency. LIF is also capable of stimulating bone formation. Furthermore, LIF exhibits activity within the nervous and immune systems. For example, LIF promotes the survival of oligodendrocytes, the cells responsible for myelinating the central nervous system (CNS). It also acts as an anti-inflammatory and analgesic cytokine with the capacity to modulate expression of the pro-inflammatory cytokine, tumor necrosis factor-α (TNF-α), as well as interleukin-6 (IL-6). LIF is, therefore, a pleiotropic molecule.

The subject inventors investigated the effects of LIF in other disease situations. Experimental autoimmune encephalomyelitis (EAE) remains the most widely studied and informative of the experimentally induced models of autoimmune disease, and is the prototype for an antigen-specific and T cell-mediated attack on the target organ. There are two types of EAE, acute and chronic relapsing, depending on the species, the age of the animal and the nature of the encephalotigenic inoculum, amongst other factors.

The pathological similarities of multiple sclerosis (MS) to EAE also point to an autoimmune pathogenesis for MS. EAE can be induced in a variety of animal species by injection of myelin basic protein (MBP) or phospholipid protein (PLP) or peptides thereof, or by adoptive transfer of MBP- or PLP-specific CD4⁺ T cells into naive recipients (1).

Acute EAE presents as a monophasic disease, characterised by inflammatory foci in the CNS with little or no demyelination. Chronic relapsing EAE (CREAE) is induced by immunisation with whole CNS tissue homogenate and, in contrast to acute EAE provoked by purified MBP and PLP, is characterized by focal demyelination in the CNS. Chronic relapsing EAE is considered to be a better animal model for MS because it induces the full spectrum of pathological changes characteristic of autoimmune demyelination (2).

The complexity of human autoimmune disease requires comparable models for a reductionist understanding of the aetiology, pathogenesis and for developing optimal treatments. More than anything else, the routine production of EAE in mice and rats using myelin proteins such as MBP and PLP, has been an important development because it has became possible to test the efficacy of potential new therapeutic agents. β-Interferon and Copolymer-1, which are now used clinically for the treatment of MS, have both been shown to ameliorate EAE in rodents.

Specifically, β-interferon can reduce the severity of EAE when administered in a delayed paradigm under certain conditions in rats (3), while Copolymer-1 is effective in preventing the induction of acute and chronic relapsing EAE induced by MBP and PLP in mice (4).

In work leading up to the present invention, the inventors determined that LIF has the capacity to abrogate experimental autoimmune encephalomyelitis. As stated above, the latter condition is an inflammatory disease of the CNS resulting from an autoimmune response to MBP or other myelin proteins including PLP. The consequence is brain and spinal cord invasion by immune cells and paralysis.

### SUMMARY OF THE INVENTION

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

One aspect of the present invention contemplates a method for the treatment and/or prophylaxis of a nervous system disease, demyelinating disease and/or an inflammatory disease condition of either the central or peripheral nervous system such as but not limited to an encephalopathic condition in a mammal, said method comprising administering to said mammal an effective amount of a polypeptide having leukemia inhibitory factor (LIF) properties or a derivative, homologue or analogue thereof for a time and under conditions sufficient to prevent or reduce onset of the condition or to ameliorate the symptoms of the condition.

Another aspect of the present invention is directed to a method for the treatment and/or prophylaxis of an encephalomyelopathic, myelopathic and/or a neuropathic condition in a mammal, said method comprising administering to said mammal an effective amount of a polypeptide having LIF properties or a derivative, homologue or analogue thereof for a time and under conditions sufficient to prevent or reduce onset of the condition or to ameliorate the symptoms of the condition.

A further aspect of the present invention contemplates a method for the treatment and/or prophylaxis of either experimental autoimmune encephalomyelitis or multiple sclerosis or a related or homologous disease condition such as a nervous system disease in a mammal, said method comprising administering to said mammal, an effective amount of LIF or a derivative, homologue or analogue thereof for a time and under conditions sufficient for the symptoms of the disease condition to be ameliorated.

Still another aspect of the present invention contemplates a method for the treatment and/or prophylaxis of multiple sclerosis or a related or homologous disease condition in a mammal, said method comprising administering to said mammal, an effective amount of LIF or a derivative, homologue or analogue thereof for a time and under conditions sufficient for the symptoms of the disease condition to be ameliorated.

Yet another aspect of the present invention extends to the use of a polypeptide having LIF properties such as LIF or a derivative, homologue or analogue thereof in the manufacture of a medicament for the treatment and/or prophylaxis of a nervous system disease, demyelinating disease and/or an inflammatory disease of either the central or peripheral nervous system such as but not limited to an encephalopathic condition such as an encephalomyelopathic condition.

Even still another aspect of the present invention extends to a composition comprising a polypeptide having LIF properties for use in the treatment and/or prophylaxis of a nervous system disease, demyelinating disease and/or an inflammatory disease of either the central or peripheral nervous system such as but not limited to an encephalopathic condition such as an encephalomyelopathic condition, said composition further comprising one or more pharmaceutically acceptable carriers and/or diluents.

Even yet another aspect of the present invention extends to an agent comprising a polypeptide having LIF properties for use in the treatment and/or prophylaxis of a nervous system disease, demyelinating disease and/or an inflammatory disease of either the central or peripheral nervous system such as but not limited to an encephalopathic condition such as an encephalomyelopathic condition, said agent further comprising one or more pharmaceutically acceptable carriers and/or diluents.

### BRIEF DESCRIPTION OF THE FIGURES

FIG. 1 is a graphical representation showing the effect of various doses of LIF and ENE severity. Mean disease scores, LIF versus vehicle. LIF was administered daily on days 0-18. Day 18 control versus LIF 25 μg/kg p<0.01. Error bars showed standard errors.

FIG. 2 is a graphical representation showing mean disease scores, LIF versus vehicle (placebo; murine serum albumin (MSA)). LIF was administered daily on days 0-18. Day 18 p<0.01, n=18 per group. Error bars show standard errors.

FIG. 3 is a graphical representation showing the cumulative probability of death following treatment with mice with LIF from day 0 to day 18.

FIG. 4 is a graphical representation showing mean disease scores, LIF versus vehicle (placebo; MSA). Mice were divided into two groups with equal disease severity on day 12. LIF was administered daily on days 12-24. Day 24 p=0.03, n=20 per group. Error bars show standard error.

FIG. 5 is a graphical representation showing the temperal cumulative probability of death in placebo (MSA) versus LIF treated animals.

FIG. 6 are two histograms showing proliferation on splenic mononuclear cells in the presence of 2, 10 and 25 μg/kg of LIF or MSA after 48 (B) and 72 (A) hrs of culture.
[symbol] =ave nil, average nil (no treatment)
[symbol] =ave PLP (4 μg), average 4 μg PLP treatment
[symbol] =ave PLP (20 μg), average 20 μg PLP treatment
[symbol] =IL-2 (100 U/ml), average 100 U/ml IL-2 treatment

FIG. 7A is a graphical representation showing the effect of various doses of LIF on EAE severity. Mean disease scores, LIF versus vehicle (MSA). LIF was administered daily on days 0-18. Day 18 control versus LIF 25 μg/kg p<0.01. Error bars show standard errors.

FIG. 7B is graphical representation showing the effect of 60 μg/kg/day LIF on EAE severity. Mean disease scores, LIF versus vehicle (MSA). LIF was administered daily on days 0-18. Error bars show standard errors.

FIG. 8 are two histograms showing proliferation assays of cultured spleenocytes. Effect of prior exposure to various concentrations of LIF for 12 days. 48 hours of culture (A) and 72 hours of culture (B).
[symbol] =no treatment
[symbol] =PLP at 4 μg
[symbol] =PLP at 20 μg
[symbol] =PLP at 20 μg

FIG. 9 is a photographic representation showing embryonic day 18 murine cortex lysates demonstrate the specificity of the polyclonal anti-LIFR-beta antibody. Knockout mice (−/−) show no band at 190 kD, and the signal obtained from heterozygotes (+/−) is approximately half the strength of that obtained from the wild-type animals (+/+). Adult cortex (A+/+) demonstrates continues expression of the receptor.

FIG. 10 is a photographic representation showing adult mouse cerebellum/brainstem lysates demonstrate the specificity of the polyclonal anti-gp130 antibody. Lysate from the relevant adult knockout mouse (gp130 STAT-3 deletion mutant, deleting the site against which the Ab is raised) shows no signal at 130 kD, in contrast to the littermate wildtype control (+/+) which is positive.

FIG. 11 is a photographic representation showing expression levels of gp130 (left) and LIFR-beta (right) from spinal cord lysates obtained from LIF and vehicle treated EAE animals compared with non-disease controls (C). Gp130 expression is invariate. EAE results in marked upregulation of LIFR-β.

FIG. 12 is a graphical representation showing expression levels of STAT-3 (top panel) and Phospho-STAT-3 (bottom panel) from the same experiment as FIG. 11. Basal STAT-3 levels are low and not upregulated in the context of early EAE. LIF treatment results in marked upregulation of STAT-3 and detectable levels of Phospho-STAT-3.

FIG. 13 is a graphical representation showing the mean disease scores from dose range finding study, LIF versus vehicle. Mice were divided into three groups with equal disease severity on day 14-16. LIF was then administered daily for 40 days. Day 56, p=0.01 control versus LIF 25 μg/kg; p=0.04 control versus LIF 10 μg/kg. Error bars show standard error.

FIG. 14 is a graphical representation showing mean disease scores from main studay, LIF versus vehicle. Mice were divided into three groups (n=15 per group) with equal disease severity on day 16-1. LIF was then demonstrated daily by subcutaneous injection for 50 days. Day 60, p=0.06 control versus LIF 25 μg/kg;. Error bars show standard error.

The following abbreviations are used in the specification:—

| Abbreviation | Definition |
|---|---|
| CNS | Central nervous system |
| CREAE | Chronic relapsing EAE |
| EAE | Experimental autoimmune encephalitis |
| ELISA | Enzyme linked immunosorbent assay |
| ES | Embryonic stem cells |
| H37Ra | Strain of Mycobacterium tuberculosis |
| IL-2 | Interleukin-2 |
| IL-6 | Interleukin-6 |
| LIF | Leukemia inhibitory factor¹ |
| MBP | Myelin basic protein |
| mcg | Microgram |
| MS | Multiple sclerosis |
| MSA | Murine serum albumin |
| PLP | Proteolipid protein |
| TNF-α | Tumor necrosis factor-α |

¹Emfilermin is the World Health Organization approved name for the pharmaceutical form of recombinant human LIF produced in E. coli in a manner that makes it suitable for human clinical use. Emfilermin is formulated in an aqueous buffer at low pH and is suitable for subcutaneous injection.

### DETAILED DESCRIPTION OF THE PREFERRED EMBODIMENTS

The present invention is predicated in part on the determination that LIF has the capacity to abrogate disease conditions of nervous systems.

Accordingly, one aspect of the present invention contemplates a method for the treatment and/or prophylaxis of a nervous system disease, demyelinating disease and/or an inflammatory disease condition of either the central or peripheral nervous system such as but not limited to an encephalopathic condition in a mammal, said method comprising administering to said mammal an effective amount of a polypeptide having leukemia inhibitory factor (LIF) properties or a derivative, homologue or analogue thereof for a time and under conditions sufficient to prevent or reduce onset of the condition or to ameliorate the symptoms of the condition.

The present invention extends to any encephalopathic, neuropathic or myelopathic condition. Such conditions may be induced by autoimmune mechanisms or may be induced by an infectious agent or chemical toxicant or induced following radiation therapy or chemotherapy or may be idiopathic. Examples of autoimmune encephalopathy, myelopathy and neuropathy include conditions exacerbated by an autoimmune response to myelin proteins. This is postulated to occur in experimental autoimmune neuritis, encephalomyelitis, multiple sclerosis and neuropathic acute demyelinating neuropathies (e.g. Guillian Barre syndrome) and chronic inflammatory demyelinating polyneuropathy. Pathogenic-induced encephalopathy includes microbial encephalitis, viral encephalitis, post-infectious encephalopathy and as well as progressive multi-focal leucoencephalopathy due to JC virus infection. The present invention also extends to genetic conditions such as leucodystrophies.

Reference herein to “nervous system disease” includes reference to encephalopathic, myelopathic or neuropathic conditions.

It is a particular aspect of the present invention to treat encephalomyelitis such as but not limited to encephalomyelitis and multiple sclerosis.

Accordingly, another aspect of the present invention is directed to a method for the treatment and/or prophylaxis of an encephalomyelopathic, myelopathic and/or a neuropathic condition in a mammal, said method comprising administering to said mammal an effective amount of a polypeptide having LIF properties or a derivative, homologue or analogue thereof for a time and under conditions sufficient to prevent or reduce onset of the condition or to ameliorate the symptoms of the condition.

A “polypeptide having leukemia inhibitory factor properties” means LIF or a derivative or homologue thereof or a fusion or hybrid protein comprising all or a portion of LIF or its derivative or homologue or an analogue of LIF. Generally, the polypeptide having LIF properties exhibits at least one activity associated with LIF and in the present case is capable of ameliorating the effects of a nervous system disease, demyelinating disease and/or an inflammatory disease of either the central or peripheral nervous system such as but not limited to an encephalomyelopathic condition such as either experimental autoimmune encephalomyelitis or multiple sclerosis.

In a particularly preferred embodiment, the polypeptide is mammalian LIF such as but not limited to human, primate, murine or livestock animal LIF. Reference herein to “LIF” or its full name “leukemia inhibitory factor” includes reference to its derivatives, homologues and analogues. LIF is well described in International Patent Application No. PCT/AU88/00093.

Generally, the present invention relates to the treatment of a disease condition which becomes apparent following the development of symptoms or through biochemical or genetic testing or following neuroimaging (e.g. MRI scan). However, where a risk has been identified of developing a nervous system disease, demyelinating disease and/or an inflammatory disease of either the central or peripheral nervous system such as but not limited to an encephalopathic condition, then the administration of a polypeptide having LIF activity including LIF is indicated to reduce the likelihood of development of the condition. A risk of development of the disease may be determined genetically such as by way of a genetic predisposition to the condition or following infection by an agent known to cause encephalitis. Accordingly, the present invention extends to the treatment and prophylaxis of a nervous system disease, demyelinating disease and/or an inflammatory disease of either the central or peripheral nervous system such as but not limited to an encephalopathic condition.

The present invention contemplates the treatment and/or prophylaxis of mammals. Reference herein to mammals includes humans, primates, livestock animals (e.g. sheep, horses, cows, pigs, donkeys), laboratory test animals (e.g. mice, rats, rabbits, guinea pigs, hamsters), companion animals (e.g. dogs, cats) and captured wild animals. In an alternative embodiment, the subject is a murine animal with, for example, experimental autoimmune encephalomyelitis. Such an animal is a useful murine model for encephalomyelopathic conditions such as multiple sclerosis.

In a particularly preferred embodiment, the present invention contemplates a method for the treatment and/or prophylaxis of either experimental autoimmune encephalomyelitis or multiple sclerosis or a related or homologous disease condition such as a nervous system disease in a mammal, said method comprising administering to said mammal, an effective amount of LIF or a derivative, homologue or analogue thereof for a time and under conditions sufficient for the symptoms of the disease condition to be ameliorated.

An example of a disease condition related or homologous to experimental autoimmune encephalomyelitis is multiple sclerosis (MS). As stated above, however, the present invention extends to all nervous system disease conditions such as acute and chronic demyelination neuropathies (e.g. Guillian Barre syndrome) or chronic inflammatory demyelinating polyneuropathy as well as central nervous system demyelination and radiation and/or chemotherapy-induced leucoencephalopathy as well as genetically determined conditions causing demyelination including but not restricted to the leucodystrophies.

Preferably, the mammal is a human or a laboratory test animal.

Accordingly, in a particularly preferred embodiment, the present invention contemplates a method for the treatment and/or prophylaxis of MS or a related or homologous disease condition in a mammal, said method comprising administering to said mammal, an effective amount of LIF or a derivative, homologue or analogue thereof for a time and under conditions sufficient for the symptoms of the disease condition to be ameliorated.

The present invention further extends to the use of a polypeptide having LIF properties such as LIF or a derivative, homologue or analogue thereof in the manufacture of a medicament for the treatment and/or prophylaxis of a nervous system disease, demyelinating disease and/or an inflammatory disease of either the central or peripheral nervous system such as but not limited to an encephalopathic condition such as an encephalomyelopathic condition.

The method and use according to these and other aspects of the present invention may comprise the administration of LIF alone or in combination with one or more other therapeutic agents such as but not limited to one or more other cytokines. The additional agents may be administered simultaneously or sequentially with LIF. Sequential administration means separate administrations within seconds, minutes, hours, days or weeks of LIF and the other agent LIF and the other agent or agents may be administered in any order. Particularly useful other agents include interferon-β, steroids, methotrexate and/or other immunosuppressive agents used in the treatment of encephalopathic conditions.

The present invention further extends to a composition comprising a polypeptide having LIF properties for use in the treatment and/or prophylaxis of a nervous system disease, demyelinating disease and/or an inflammatory disease of either the central or peripheral nervous system such as but not limited to an encephalopathic condition such as an encephalomyelopathic condition, said composition further comprising one or more pharmaceutically acceptable carriers and/or diluents.

Reference to a composition includes reference to an agent.

Accordingly, the present invention further extends to an agent comprising a polypeptide having LIF properties for use in the treatment and/or prophylaxis of a nervous system disease, demyelinating disease and/or an inflammatory disease of either the central or peripheral nervous system such as but not limited to an encephalopathic condition such as an encephalomyelopathic condition, said agent further comprising one or more pharmaceutically acceptable carriers and/or diluents.

Preferably, the composition is exclusively used for the treatment and/or prophylaxis of the conditions.

Pharmaceutically acceptable carriers and/or diluents include any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, use thereof in the therapeutic compositions is contemplated. Supplementary active ingredients can also be incorporated into the compositions.

The pharmaceutical composition may also comprise genetic molecules such as a vector capable of transfecting target cells where the vector carries a nucleic acid molecule capable of modulating expression of a nucleic acid molecule encoding binding partner. The vector may, for example, be a viral vector. In this regard, a range of gene therapies are contemplated by the present invention including isolating certain cells, genetically manipulating and returning the cell to the same subject or to a genetically related or similar subject. The present invention further contemplates the administration of “naked” DNA which encodes a LIF polypeptide or an agonist of expression of a LIF gene.

LIF may be administered in any number of ways including via intravenous, intraperitoneal, subcutaneous, intrathecal, rectal, intranasal or aerosol administration. Prolonged infusion or sustained release administration is also contemplated. Preparations comprising LIF can be conveniently prepared with reference to Remington's Pharmaceutical Sciences, Mack Publishing Company, Easton, Pa., U.S.A.

As stated above, the present invention extends to LIF and its derivatives, homologues and analogues. A derivative includes a mutant, fragment, part, portion or region of LIF such as a single or multiple amino acid substitution, addition and/or deletion to the LIF amino acid sequence. A derivative also includes hybrid molecules and fusion molecules such as between LIF polypeptides from different species of animals or between polymorphic variants of LIF polypeptides within the one species.

Other derivatives of LIF contemplated by the present invention include a range of glycosylation variants from a completely unglycosylated molecule to a modified glycosylated molecule. Altered glycosylation patterns may result from expression of recombinant molecules in different host cells.

A “homologue” includes a LIF molecule from a different animal species as well as a structurally and/or functionally related molecule from the same species. A polymorphic variant is regarded herein as a homologue.

LIF “analogues” contemplated herein include but are not limited to modifications to side chains, incorporating of unnatural amino acids and/or their derivatives during peptide, polypeptide or protein synthesis and the use of crosslinkers and other methods which impose conformational constraints on the proteinaceous molecule or their analogues. Analogues may exhibit greater stability, longer serum half-life and enhanced efficacy.

Examples of side chain modifications contemplated by the present invention include modifications of amino groups such as by reductive alkylation by reaction with an aldehyde followed by reduction with NaBH₄; amidination with methylacetimidate; acylation with acetic anhydride; carbamoylation of amino groups with cyanate; trinitrobenzylation of amino groups with 2, 4, 6-trinitrobenzene sulphoric acid (TNBS); acylation of amino groups with succinic anhydride and tetrahydrophthalic anhydride; and pyridoxylation of lysine with pyridoxal-5-phosphate followed by reduction with NaBH₄.

The guanidine group of arginine residues may be modified by the formation of heterocyclic condensation products with reagents such as 2,3-butanedione, phenylglyoxal and glyoxal.

The carboxyl group may be modified by carbodiimide activation via O-acylisourea formation followed by subsequent derivitization, for example, to a corresponding amide.

Sulphydryl groups may be modified by methods such as carboxymethylation with iodoacetic acid or iodoacetamide; performic acid oxidation to cysteic acid; formation of a mixed disalphides with other thiol compounds; reaction with maleimide, maleic anhydride or other substituted maleimide; formation of mercurial derivatives using 4-chloromercuribenzoate, 4-chloromercuriphenylsulphonic acid, phenylmercury chloride, 2-chloromercuri-4-nitrophenol and other mercurials; carbamoylation with cyanate at alkaline pH.

Tryptophan residues may be modified by, for example, oxidation with N-bromosuccinimide or alkylation of the indole ring with 2-hydroxy-5-nitrobenzyl bromide or sulphenyl halides. Tyrosine residues on the other hand, may be altered by nitration with tetranitromethane to form a 3-nitrotyrosine derivative.

Modification of the imidazole ring of a histidine residue may be accomplished by alkylation with iodoacetic acid derivatives or N-carbethoxylation with diethylpyrocarbonate.

Examples of incorporating unnatural amino acids and derivatives during peptide synthesis include, but are not limited to, use of norleucine, 4-amino butyric acid, 4-amino-3-hydroxy-5-phenylpentanoic acid, 6-aminohexanoic acid, t-butylglyceine, norvaline, phenylglycine, orithine, sarcosine, 4-amino-3-hydroxy-6-methylheptanoic acid, 2-thienyl alanine and/or D-isomers of amino acids.

Crosslinkers can be used, for example, to stabilize 3D conformations, using homo-bifunctional crosslinkers such as the bifunctional imido esters having (CH₂)ₙ spacer groups with n=1 to n=6, glutaraldehyde, N-hydroxysuccinimide esters and hetero-bifunctional reagents which usually contain an amino-reactive moiety such as N-hydroxysuccinimide and another group specific-reactive moiety such as maleimido or dithio moiety (SH) or carbodiimide (COOH). In addition, peptides can be conformationally constrained by, for example, incorporation of C_{α} and N_{α}-methylamino acids, introduction of double bonds between C_{α} and C_{β} atoms of amino acids and the formation of cyclic peptides or analogues by introducing covalent bonds such as forming an amide bond between the N and C termini, between two side chains or between a side chain and the N or C terminus.

All derivatives, homologues and analogues of LIF are encompassed by the terms “leukemia inhibitor factor”, “LIF”, “LIF polypeptide” and a “polypeptide having LIF properties”.

The effective amount of LIF contemplated for use in accordance with the subject method in the amount required to ameliorate the symptoms of the encephalopathic condition. Suitable amounts may need to be varied according to the condition and severity of the condition being treated. Multiple doses may be administered or a single bolus may be given. Examples of effective amounts include from about 10 ng/kg body weight to about 10 mg/kg body weight and more particularly from about 0.1 μg/kg body weight to about 5 mg/kg body weight and even more particularly from about 0.5 μg/kg body weight to about 1 mg/kg body weight. Administration may be per hour, per day, per week, or per month.

Generally, recombinant LIF is administered. For the treatment of humans, for example, recombinant human LIF is preferred although the present invention extends to humanized forms of non-human LIF.

The present invention is further described by the following non-limiting Examples.

### EXAMPLE 1

### Effect of LIF on Experimental Autoimmune Encephalomyelitis-I

Female 7-10 week old SL/J mice were immunized with 100 μg of serine-substituted peptide from mouse PLP (amino acids 139-151) dissolved in 0.1 ml phosphate-buffered saline (PBS) emulsified with an equal volume of complete Freund's adjuvant, containing Mycobacterium tuberculosis H37Ra (4 mg/ml). Mice were also given 400 ng of pertussis toxin into the tail vein on the day of inoculation and 3 days later. This particular model results in severe monophasic EAE. Murine LIF in 0.3% v/v mouse serum albumin [MSA], pH 7.4. (Sigma) was intraperitoneally administered daily on day 0-18. The initial study was conducted to determine approximate dose ranges. Three cohorts of mice received 2, 10 or 25 μg/kg of LIF, respectively and mice injected with MSA alone served as controls. The severity of disease was evaluated daily using the following scale: 0, no clinical symptoms; 1, fur ruffling and/or distal tail weakness; 2, tail atonia and/or slight hind limb paralysis; 3, complete paralysis affecting both hind limbs; 4 complete paralysis of both hind limbs and fore-limb weakness. Statistical comparisons between groups were performed using the unpaired two-tailed Student's t-test.

Splenic mononuclear cell suspensions were prepared from mice 12 days after inoculation. As an indirect marker of lymphocyte activation, the proliferation index of the cells was assessed in either PLP peptide (4 or 20 μg/ml) or interleukin-2 (20 IV/ml) by measuring 3H-thymidine incorporation after a 2 day culture period and a 16 hour pulse. The ratio of CD4 to CD8 positive cells in the spleen was assessed at the same timepoint by FACS.

Western blot of immunoprecipitate of CNS lysates was performed according to standard methods. The gp130 antibody used was the polyclonal rabbit-anti-mouse sc-656 antibody (Santa Cruz). Specificity of the signal was determined by the use of CNS tissue from a mouse in which the gp130 receptor has been truncated as a control. The truncation removes the domain against which the antibody was raised.

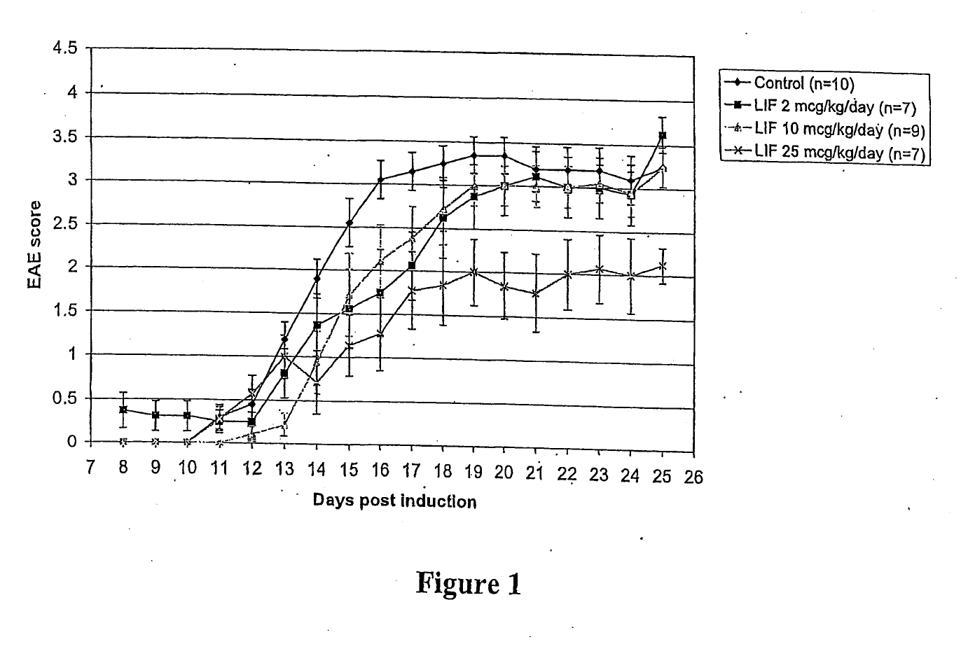

The groups of mice receiving 25 μg/kg of LIF (n=7) showed a reduction of experimental autoimmune encephalomyelitis (EAE) disease score compared with the placebo groups (n=10). On day 18 post-induction, the mean disease score in the LIF treated mice was 1.8 compared with 3.2 in the placebo cohort (p<0.01) [FIG. 1]. The death rate at day 18 was 67% in the placebo group, compared with 33% in the LIF treated group. Animals treated with lower doses of LIF (2 μg/kg and 10 μg/kg) showed no significant disease reduction (FIG. 1). LIF did not retard the onset of disease.

Since the initial study produced promising results, the effect of LIF in this model of MS was then examined in greater detail. This focused on strengthening the data in the high dose LIF group.

Two cohorts of mice were used to confirm the above data which showed that high dose LIF (25 μg/kg/day) was able to slow the progression of EAE when administered from day 0-18. This was performed as described in the previous section for the pilot study.

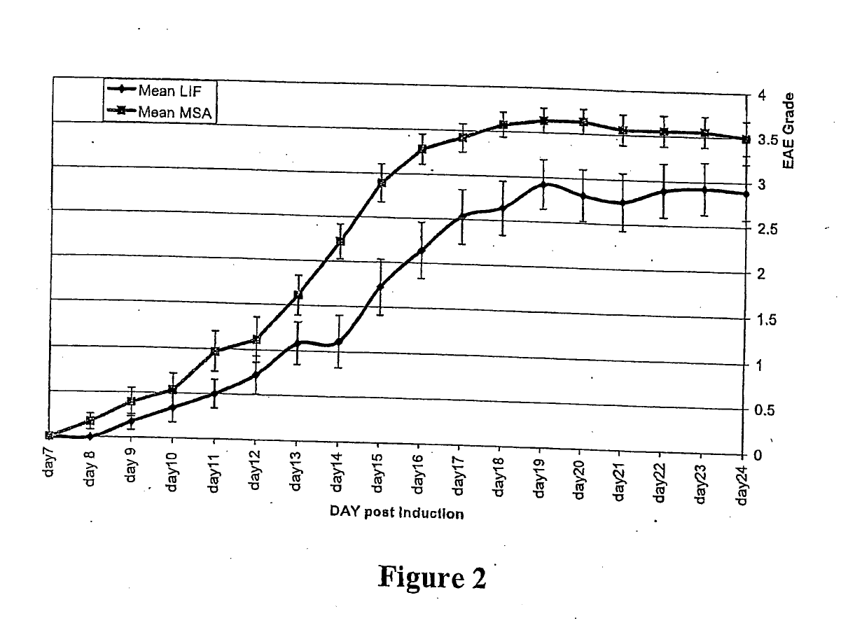

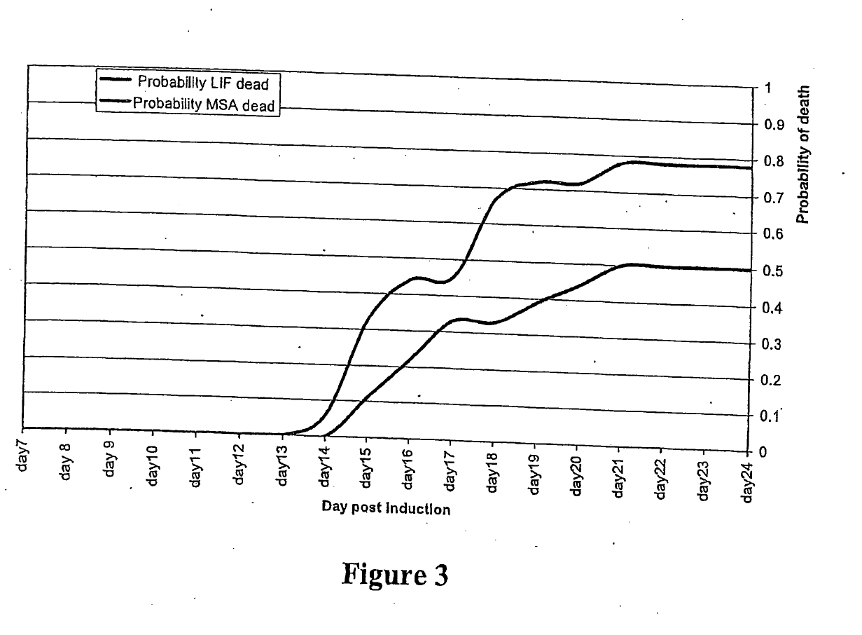

The cohort that received LIF exhibited a significant reduction in disease score from day 18 when compared to the vehicle group [FIG. 2]. On day 18, the average disease score in the LIF treated group was 2.6 compared with 3.6 in the vehicle group p<0.01). By day 24, 78% of the animals receiving vehicle had died, compared with 50% of LIF treated animals. This is shown in the cumulative probability of death shown in FIG. 3.

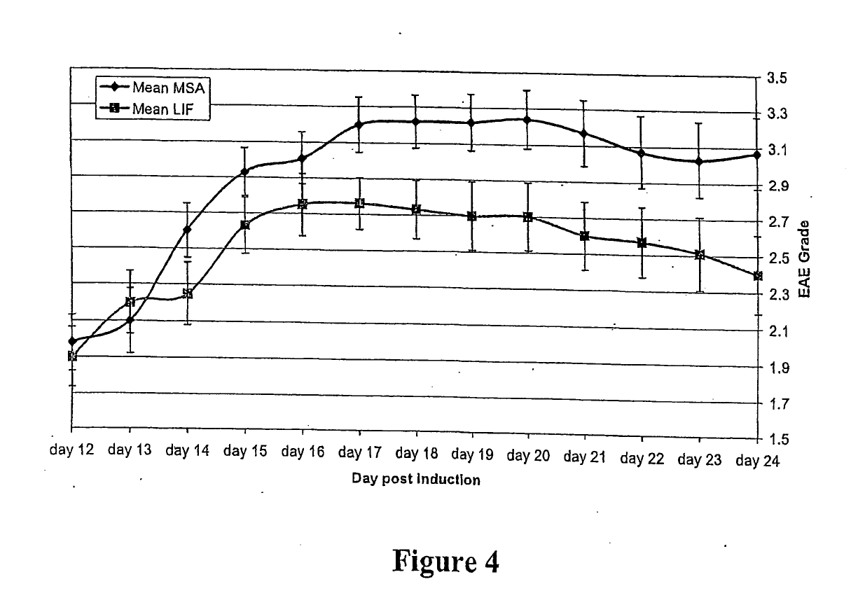

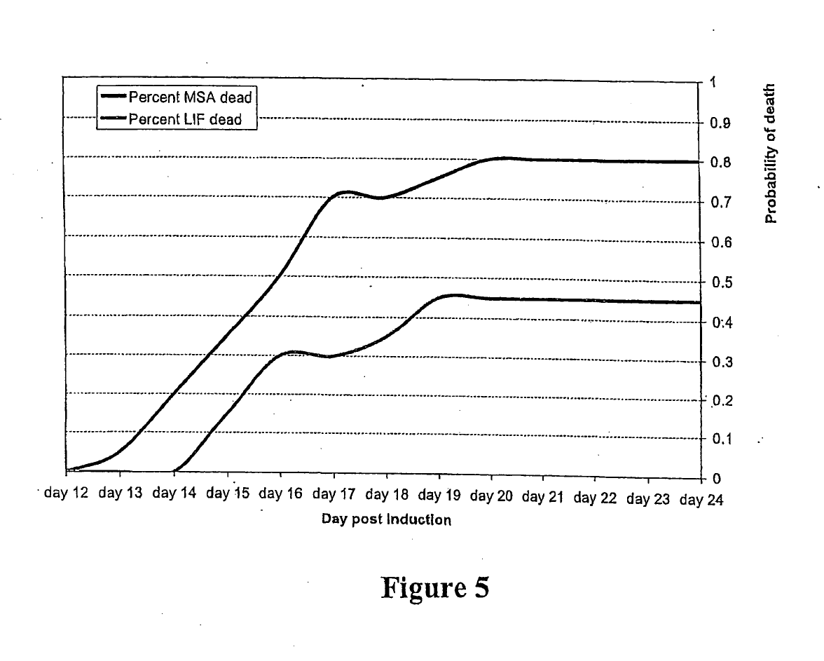

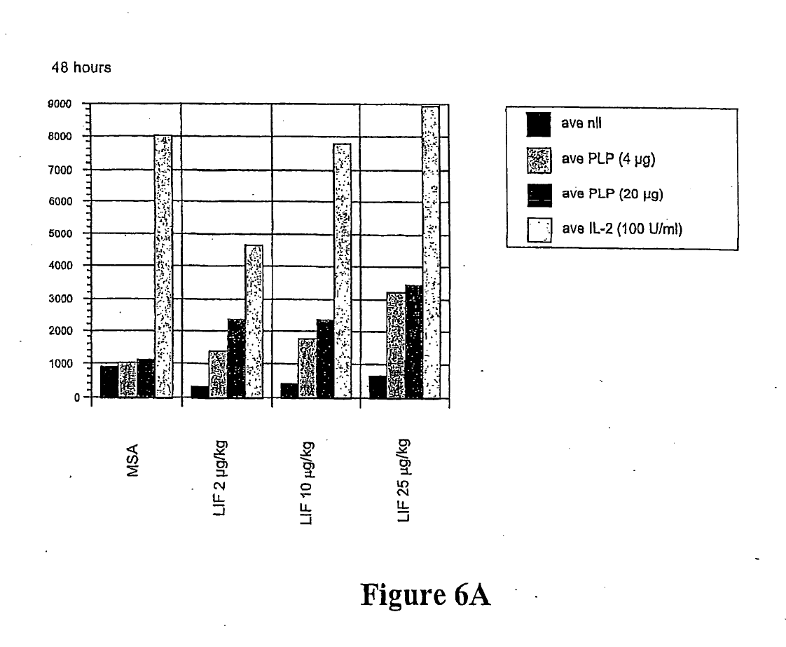

A further two cohorts of mice were utilized in order to determine whether LIF at 25 μg/kg/day given after disease onset could alter the subsequent course of disease. Inoculated animals were randomized on day 12 into two groups (total n=29 in each group), matched on their neurological grade and the amount of weight loss in the preceding 24 hours. LIF (25 μg/kg) or MSA were administered IP from day 12 to 24. Matching by grade was successful as indicated by a nearly identical average EAE score on the first day after matching (day 13). The cohort that received LIF exhibited a significant reduction in disease score from day 17. On day 24, the average disease score in the LIF treated group was 2.4, compared with 3.1 in the placebo group (p<0.03) [FIG. 4]. By day 24, 80% of placebo animals had died, compared with 45% of LIF treated animals [FIG. 5].

### EXAMPLE 2

### Immunological Assessments

The percentage of splenic white cells expressing CD4 and CD8 collected on day 12 after disease induction, as assessed by FACS sorting, were similar in MSA and LIF treated animals, at all doses of LIF examined (2, 10, 25 μg/kg).

Thymidine incorporation assays of the same spleen cells yielded potentially interesting results at a culture density of 2×10⁵ cells/well, at both 48 and 72 hours of culture. Specifically, there was a reduction of non-selective proliferation (IL-2 induced) of splenic cells collected from animals exposed to low dose LIF compared with those exposed to either MSA or higher doses of LIF. However, there was a trend towards enhanced proliferation in response to PLP (the induction antigen) in the animals treated with LIF, in a dose-dependent manner [FIG. 6]. These results are further analyzed (a) confirm the above results by repeating the experiment and (b) to measure cytokine profiles of the proliferating cells in response to PLP stimulation. This enables characterization of the response as pro-inflammatory or anti-inflammatory. However, it is likely that LIF exerts some effect upon splenic mononuclear cells. The clinical data show that this effect is not deleterious in the context of EAE.

### EXAMPLE 3

### Expression of the LIF Receptor Components GP130 and LIF Receptor β in the CNS

The inventors have demonstrated definite expression of the gp130 component of the receptor in the adult murine cerebellum and brainstem by Western blot immunoprecipitation. The gp130 component cannot be visualized by a straight Western blot of lysate, but is easily demonstrated by immunoprecipitation. Results indicated that there was no change in the amount of gp130 expression in EAE animals compared with controls.

Results suggested that the LIF-receptor β component is also detectable by immunoprecipitation Western blot.

### EXAMPLE 4

### Delayed Administration of LIF

The experiment outlined in Example 1 is repeated to confirm the efficacy of delayed administration. Similar results as outlined in the third cohort of mice in Example 1 are anticipated.

### EXAMPLE 5

### Cytokine Production

Splenic mononuclear cells are removed at day 12 post-induction of experimental autoimmune encephalomyelitis and cultured. ELISA and/or other immunodiagnostic procedures are then used to determine the cytokine profile. Of particular interest is the determination of whether levels of interferon-γ or IL-6 change. This enables the potential effects of LIF on the cytokine profile to be determined.

### EXAMPLE 6

### Oligodendrocytes

The numbers of oligodendrocytes in experimental autoimmune mice are determined relative to control mice in the lumbar spinal cord. A difference would indicate that LIF has an effect on oligodendrocyte survival and/or regeneration.

### EXAMPLE 7

### LIF Receptor

Murine CNS tissue from experimental autoimmune encephalomyelitis relative to control mice is subjected to Gp130 staining. This enables an assessment to be made of the potential upregulation of the LIF receptor signaling components in the context of EAE, providing a potential mechanism by which the therapeutic effects of LIF might be mediated in the context of CNS inflammatory disease.

### EXAMPLE 8

### Combination Therapy

The experiment outlined in Example 1 is repeated with LIF and interferon-β being administered simultaneously and sequentially in either order. Greater efficacy for the combination than with either agents alone is indicative of synergy and the potential value of combination therapy.

### EXAMPLE 9

### Effect of LIF on Experimental Autoimmune Encephalomyelitis-II

The study described in Example 1 was then extended to test a higher dose of LIF. Two groups of mice were used; one received LIF at 60 μg/kg/day, while the second served as the control group.

Female 7-14 week old SJL/J mice were immunized with 100 μg of serine-substituted peptide from mouse PLP (amino acids 139-151) dissolved in 0.1 ml of phosphate-buffered saline emulsified with an equal volume of complete Freund's adjuvant, containing Mycobacterium tuberculosis E37Ra (4 mg/ml). Mice were also given 400 ng of pertussis toxin into the tail vein on the day of inoculation and 3 days later. This particular model resulted in severe monophasic EAE.

Murine LIF in 0.3% w/v MSA (Sigma) was administered daily intraperitoneally on day 0-18. The severity of disease (clinical score) was evaluated daily using the following scale:

| | |
|---|---|
| 0 | no clinical symptoms; |
| 1 | fur ruffling, and/or distal tail weakness; |
| 1.5 | tail weakness and slight hindlimb weakness; |
| 2 | tail atonia, and/or slight hind limb paralysis; |
| 2.5 | tail atonia and significant incomplete hindlimb weakness; |
| 3 | complete paralysis affecting both hind limbs; |
| 4 | complete paralysis of both hind-limbs and fore-limb weakness. |

Statistical comparisons between groups were performed using the unpaired two-tailed Student's t-test.

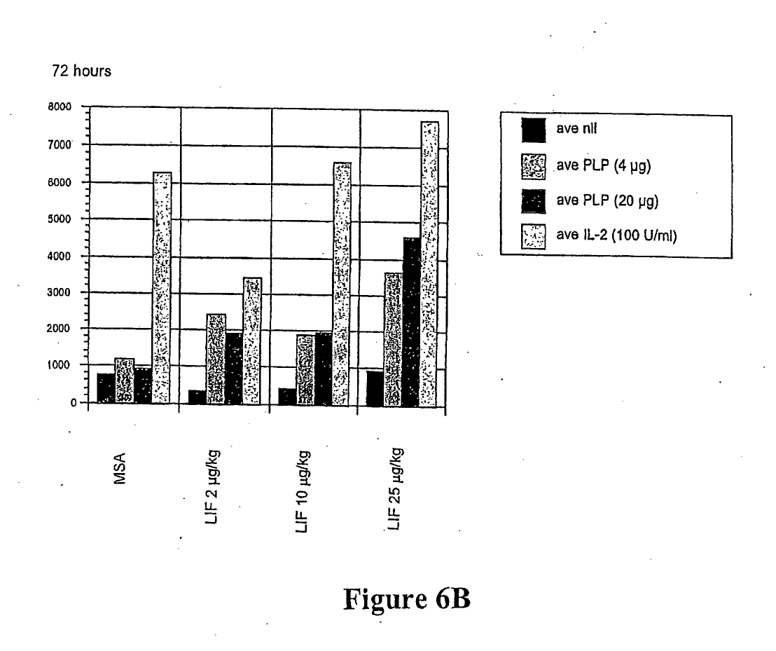

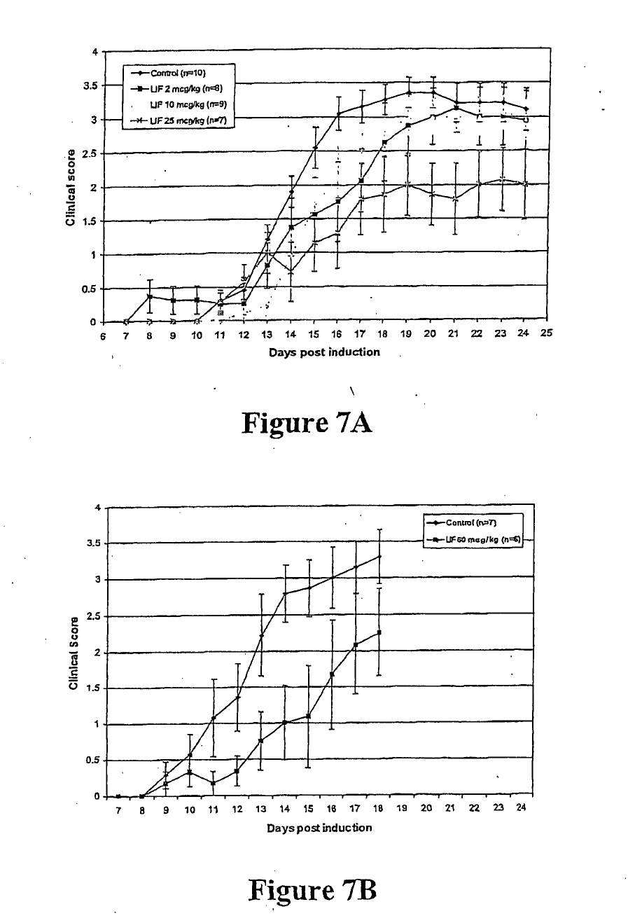

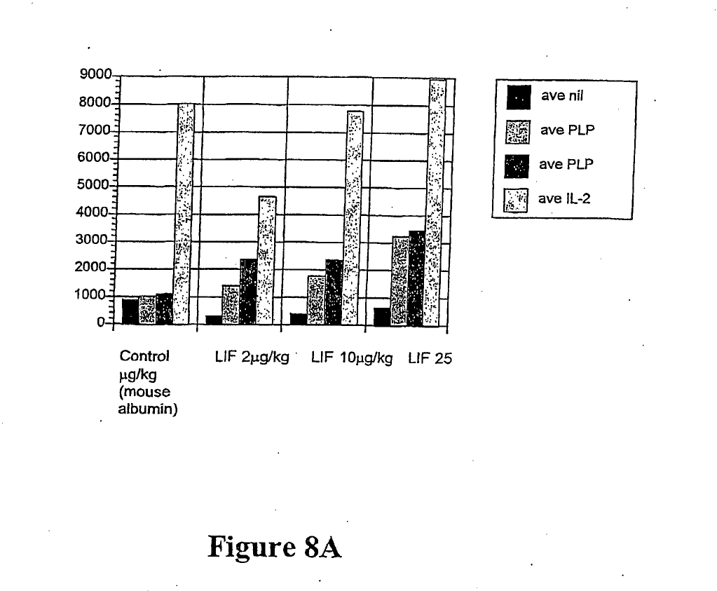

The group of mice receiving 25 μg/kg of LIF (n=7) again showed a reduction of EAE disease score compared with the placebo (MSA) groups (n=10). On day 18 post-induction, the mean disease score in the LIF treated mice was 1.8 compared with 3.2 in the placebo (MSA) cohort (p<0.01) [FIG. 7A]. The death rate at day 18 was 67% in the placebo (MSA) group, compared with 33% in the LIF treated group. Animals treated with lower doses of LIF (2 μg/kg and 10 μg/kg) showed no significant disease reduction. LIF did not retard the onset of disease. The mice that received LIF at 60 μg/kg/day also showed a significant reduction in EAE (clinical) score. However, this group performed no better than the group that received LIF at 25 μg/kg [FIG. 7B].

### EXAMPLE 10

### Immunological Assessments from Example 9

An immunological assessment from the mice of Example 9 was tested as described in Example 2.

The percentage of splenic white cells expressing CD4 and CD8 collected on day 12 after disease induction, as assessed by FACS sorting, were similar in MSA and LIF treated animals at all doses of LIF examined (2, 10, 25 μg/kg). After 72 hours of culture, splenocytes derived from LIF treated animals showed a non-significant reduction in total proliferation index, correlating with a 20% reduction in both CD4 and CD8 lymphocyte contents.

Cytokine levels of interferon-γ, interleukin-12, interleukin-5 and interleukin-10 in mixed splenocyte cultures were assessed by ELISA. Production of these cytokines were similarily all modestly reduced in the cultures derived from LIF-treated animal compared with the vehicle treated group. The results are shown in FIG. 8.

### EXAMPLE 11

### Expression of LIF Receptor and the STAT13 Signaling Molecule in the CNS from Example 9

For assessment of potential central actions of LIF, animals which had received 25 μg/kg of LIF daily from day 0 were killed on day 12 (day of overt disease onset). Spinal cords were obtained by saline-flush. Immunoprecipitation (IP) of lysed spinal cord was performed with Protein G beads and the samples were liberated from the beads under reducing conditions. They were then run on 4-20% w/v polyacrylamide gels. The gels were transferred onto appropriate membranes and Western blotting with chemiluminescent detection was used to assess for the presence and levels of components of the cognate LIF receptor, namely gp130 and LIF receptor-β. Both polyclonal rabbit-anti-mouse antibody used were tested for specificity using the relevant deletion mutants. The inventors also assessed the levels of the major downstream signaling protein activated by the LIF receptor complex, STAT3, and its activated form, TYR-phosphorylated STAT3, by immunoprecipitation/Western blotting.

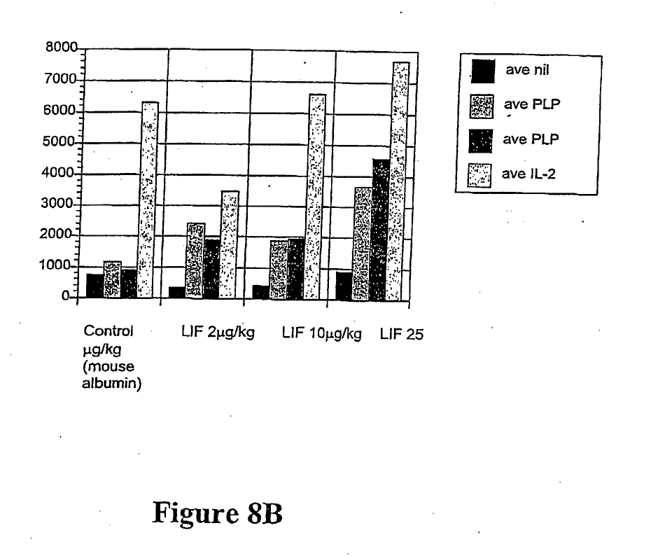

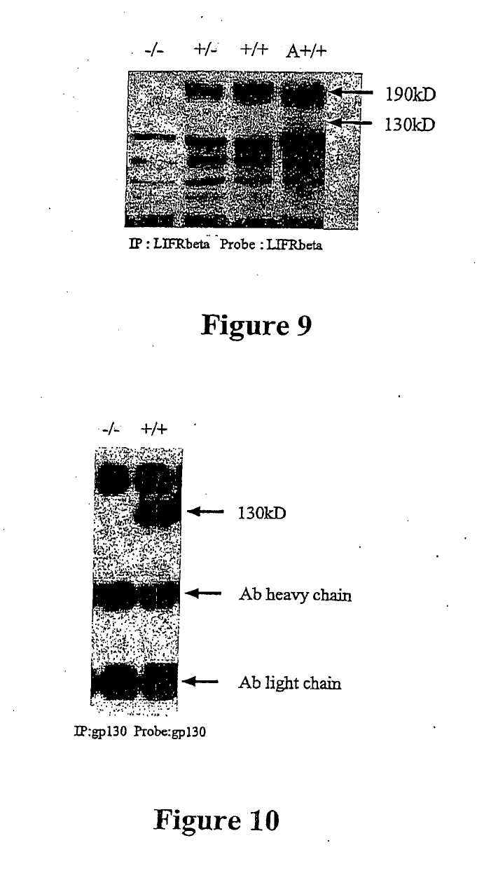

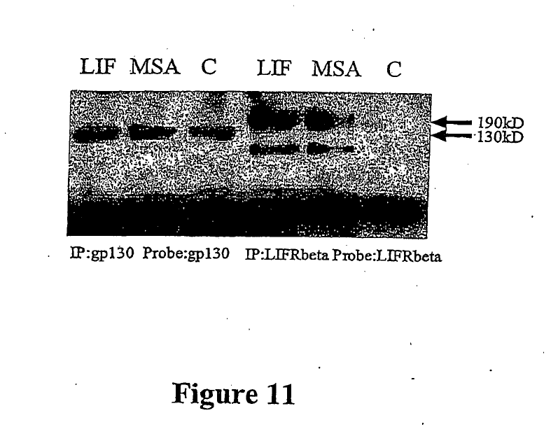

Gp130 and LIFR-beta polyclonal antibody were specific, as demonstrated by analysis of the relevant knock-out CNS tissues [FIGS. 9 and 10]. LIFR-β could not be demonstrated in the control spinal cord on this occasion, but EAE resulted in marked upregulation of the LIFR-β component. [FIG. 11] Gp130 was present in normal adult murine spinal cord, and levels did not change with EAE induction or LIF treatment. Data from subsequent experiments suggest that, under control conditions, a basal LIFR-β expression is sometimes detectable in the adult murine spinal cord, but LIFR-β levels are always upregulated in EAE.

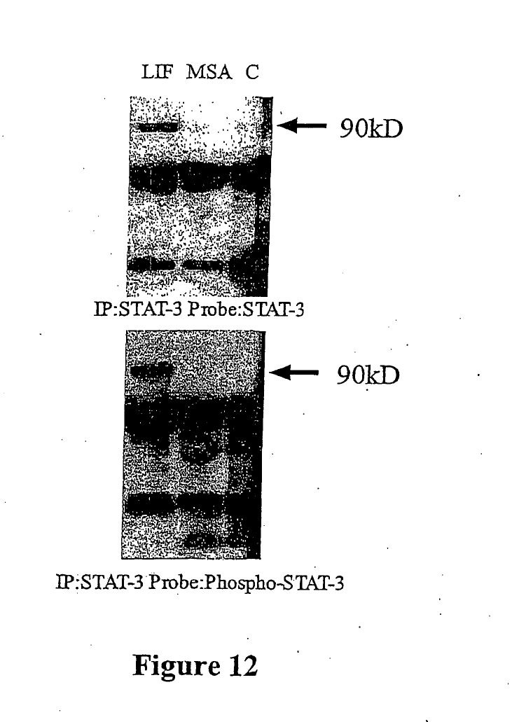

LIF administration results in marked upregulation of STAT-3 levels in the spinal cord. Basal levels were low and not changed in EAE animals treated with vehicle. Additionally, STAT-3 activation as determined by detectable levels of phosphorylated STAT-3 in the cord were detected only in animals treated with LIF [FIG. 12].

### EXAMPLE 12

### Effect of LIF on Experimental Autoimmune Encephalomyelitis-III

LIF was further tested for its capacity to abrogate experimental autoimmune encephalomyelitis in the murine model of multiple sclerosis.

An initial study was again conducted as a dose range finding study and examined the effect of 2 different doses of LIF. Two cohorts of mice received 10 or 25 μg/kg of LIF respectively and mice injected with MSA (LIF was formulated in PBS pH 7.4+0.1% mouse albumin which served as a carrier protein) alone served as controls.

Female 8-10 week old C57BL/6 mice were injected subcutaneously into one hind footpad and into the back of the neck (50 μl each) with 100 μg of peptide from mouse myelin oligodendrocyte protein [MOG] (amino acids 35-55), dissolved in 50 μl of PBS and emulsified with an equal volume of complete Freund's adjuvant containing Mycobacterium tuberculosis (4 mg/ml). Mice were then injected intravenously with pertussis vaccine (300 ng in 0.3 m 1 PBS). Pertussis injection was repeated 48 hr later. This particular model results in a chronic progressive EAE.

Murine LIF in 0.1% w/v MSA (Sigma) was administered daily subcutaneously for 40 days, beginning at the time clinical symptoms become evident, usually at day 14-16. Clinical impairment was graded daily using the following scale:

| | |
|---|---|
| 0 | no detectable impairment |
| 1 | limp tail and loss of weight |
| 2 | weakness of hindlimbs |
| 3 | complete paralysis of one/both hind limbs |
| 4 | complete paralysis of one/both hind-limbs and ascending paralysis. At |
| | this stage mice are considered moribound and must be euthanased |
| 5 | deceased |

Statistical comparisons between groups were performed using the unpaired two-tailed Student's t-test.

As this was a preliminary study, only five animals were used per group. Fourteen out of 15 mice developed clinical signs of EAE after-immunization and were used in the study. Five mice were assigned to each of the two LF treatment groups and 4 mice were used in the control group. One mouse from the high dose LIF group died on day 28 and was excluded from the analysis from that point onwards. The death was not considered treatment or disease related.

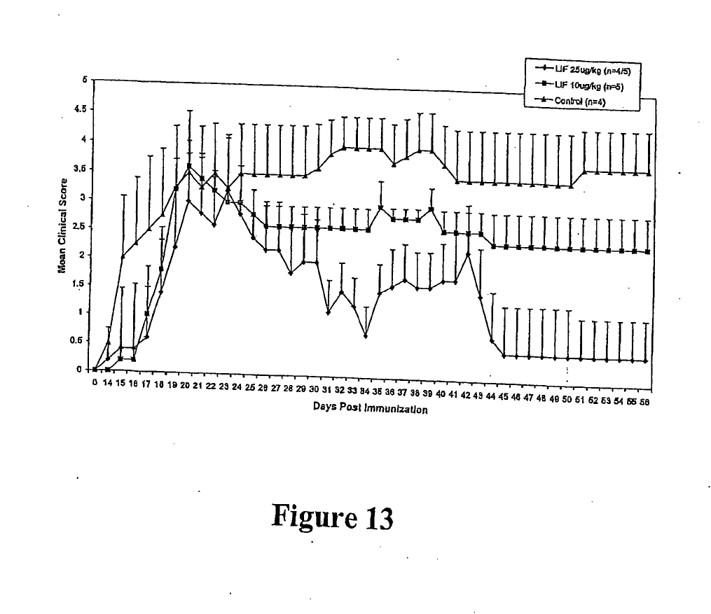

The group of mice receiving 25 μg/kg of LIF showed a reduction of EAE disease score compared with the vehicle group. On day 56 post-induction the mean disease score in the high dose LIF group was 0.4 compared with 1.3 in the vehicle cohort (p=0.01) [FIG. 13]. Mice treated with LIF at 10 μg/kg also showed a reduction in mean clinical score compared to the control group which was also significant (p=0.04). The mortality at day 56 was 50% in the control group, while no mice had died in either of the two LIF treatment groups.

Since the above study again produced promising results, the effect of LIF in this model of EAE was then examined in greater detail. This focused on strengthening the data in both the 10 and 25 μg/kg dose groups. Inoculated animals were randomised on day 16-18 into two groups (total n=15 in each group), matched on their neurological grade. LIF 10 and 25 μg/kg or vehicle (MSA) was then administered subcutaneously once daily for 50 days.

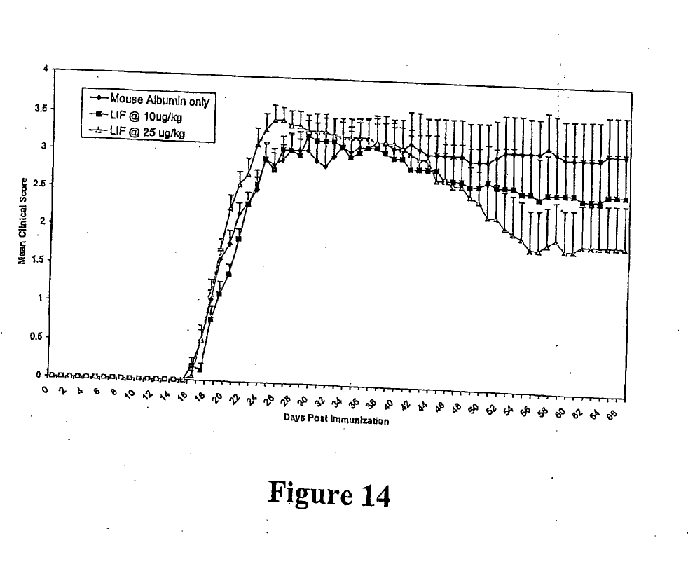

The cohort that received LIF at a dose of 25 μg/kg exhibited a significant reduction in disease score, with the effect being observed beginning at about day 40. By day 65, the average disease score in the high dose LIF group was 1.9 compared with 3.1 in the vehicle treated group (p=0.06). The cohort that received LIF at 10 μg/kg also showed a reduction in mean clinical score compared to control group. However, this was less significant than the high dose LIF group [FIG. 14].

There was also a dose-related reduction in mortality observed. On day 65, 40% of the mice in the vehicle group had died. The mortality was reduced to 33% in the low dose LIF group, while only 20% of the mice in the high dose group had died by the end of the study.

At the termination of the study the mice were killed, blood was sampled and the spleens removed. Antibody activity to MOG or MOG peptide is measured in serum by ELISA according to standard protocols. Spleenocytes are cultured and assayed for T-cell, antibody and cytokines responses. Brain and spinal cord are also removed for histological analysis to assess the degree of inflammation, demyelination and/or remyelination.

### EXAMPLE13

### Leukaemia Inhibitory Factor Reduces the Severity of Murine Experimental Autoimmune Encephalomyelitis

The potential of LIF as a treatment for the murine model of inflammatory central nervous system disease was further tested.

SJL/J mice were immunised with an encephalitogenic portion of mouse PLP in complete Freund's adjuvant. Murine LIF was administered daily on days 0-18, in doses of 2, 10, 25 and 60 μg/kg to determine the optimum dose. Subsequently, LIF (25 m/kg) was administered on days 0-18 or days 12-24 (delayed treatment groups were matched for grade of disease and weight loss), whilst control mice were injected with albumin. The relapse rate was measured up to day 60 post induction in mice treated with LIF (25 μg/kg/day) between either day 12-24 or day 12-60, and with placebo. Disease was scored using a non-parametric scale. For immunological assessments, splenic mononuclear cell suspensions were prepared from mice 12 days after inoculation, cultured at equal density for 72 hours in RPMI and the proliferation index of the cells was assessed in either PLP peptide, interluekin-2, or both, utilizing triturated thymidine incorporation. Lymphocyte numbers and the CD4/CD8 T-lymphocyte ratio in the spleen were assessed at day 12 by fluorescence-activated cell sorting, and also after 72 hour culture. Cytokine levels of interferon-γ, interleukin-12, interleukin-5 and interleukin-10 in mixed splenocyte cultures were assessed by ELISA.

For assessment of potential central actions of LIF, animals which had received 25 μg/kg of LIF daily from day 0 were killed on day 12 (day of overt disease onset). Spinal cords were obtained by saline-flush. Immunoprecipitation (IP) of lysed spinal cord was performed with Protein G beads and the samples were liberated from the beads under reducing conditions. They were then run on 4-20% w/v polyacrylamide gels. The gels were transferred on to appropriate membranes and Western blotting with chemiluminescent detection was used to assess for the presence and levels of components of the cognate LIF receptor, namely gp 130 and LIF receptor-β. Both polyclonal rabbit-anti-mouse Ab used were tested for specificity using the relevant deletion mutants. The subject inventors also assessed the levels of the major downstream signaling protein activated by the LIF receptor complex, STAT3, and its activated form, TYR-phosphorylated STAT3, by immunoprecipitation/Western blotting.

The optimum dose of LIF was 25 μg/kg/day. Mice receiving this dose of LIF from day 0-18 had abrogated disease, such that on day 18, the mean disease score was 2.6 compared with 3.6 in the placebo cohort (p<0.01). LIF did not retard the time of onset of disease. In the delayed treatment group, LIF reduced mean disease grade on day 24 from 3.1 to 2.4 (p=0.03). In contrast, the relapse rate was not affected by treatment. The numbers of splenic CD4 and CD8 positive cells were similar in both LIF-treated and control groups. After 72 hours of culture, splenocytes derived from LIF treated animals showed a non-significant reduction in total proliferation index, correlating with a 20% reduction in both CD4 and CD8 lymphocyte contents. Production of interferon-γ, IL-5, and IL-10 were similarily all modestly reduced in the cultures derived from LIF-treated animals compared with placebo.

Gp130 and LIFR-β polyclonal Ab were specific, as demonstrated by analysis of the relevant knock-out CNS tissues. LIFR-β could not be demonstrated in the control spinal cord on this occasion, but EAE resulted in marked upregulation of the LIFR-β component. Gp130 was present in normal adult murine spinal cord, and levels did not changed with EAE induction or LIF treatment. Data from subsequent experiments suggest that, under control conditions, a basal LIFR-β expression is sometimes detectable in the adult murine spinal cord, but LIFR-β levels are always upregulated in EAE.

LIF administration results in marked upregulation of STAT-3 levels in the cord. Basal levels are low and not changed in EAE animals treated with placebo (MSA). Additionally, STAT-3 activation as determined by detectable levels of phosphorylated STAT-3 in the cord can be shown only in animals treated with LIF.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations of any two or more of said steps or features.

### BIBLIOGRAPHY

1. Bernard, C., Mandel, T. and Mackay, I R, Experimental models of human autoimmune disease: overview and prototypes, in The autoimmune diseases II, N.a.M. Rose, I R Editor. 1992, Academic Press: Sau Diego. p. 47-106

2. Raine, C., Demyelinating diseases, in Textbook of Neuropatholgy, Ra.R Davis, D M, Editor. 1990, Williams and Wilkins: Baltimore. p. 535-620

3. Ruuls, S. R., de Labie, M. C., Weber, K. S., Botman, C. A., Groenstein, R J., Dijkstra, C. D., Olsson, T., van der Meide, P. H., J. Immunol. 157(12):5721-31, 1996

4. Aharoni, R., Sela, M., Teitelbaum, D., J. Neurol. 243(4 Suppl. 1): p. S8-13, 1996

What is claimed is:

1. A method for the treatment of a demyelinating disease condition of either the central or peripheral nervous system in a mammal, wherein the disease condition is selected from the group consisting of experimental autoimmune neuritis, encephalomyelitis, neuropathic acute demyelinating neuropathy and chronic inflammatory demyelinating polyneuropathy, said method comprising administering to said mammal an effective amount of a polypeptide having leukemia inhibitory factor (LIF) properties for a time and under conditions sufficient to ameliorate the symptoms of the condition.

2. A method according to claim 1 wherein the disease condition of the central or peripheral nervous system is an encephalopathic, neuropathic or myelopathic condition.

3. A method according to claim 2 wherein the encephalopathic, neuropathic or myelopathic condition is an autoimmune encephalopathic, neuropathic or myleopathic condition.

4. A method according to claim 3 wherein the autoimmune condition is exacerbated by an immune response to myelin proteins.

5. A method according to claim 2 wherein the disease condition is encephalomyelitis.

6. A method according to claim 1 wherein the encephalopathic condition is pathogen-induced encephalopathy.

7. A method according to claim 6 where the pathogen-induced encephalopathy is microbial encephalitis, viral encephalitis, post-infectious encephalopathy or progressive multi-focal leucoencephalopathy.

8. A method for the treatment of experimental autoimmune encephalomyelitis or a related or homologous disease condition in a mammal, said method comprising administering to said mammal, an effective amount of LIF for a time and under conditions sufficient for the symptoms of the disease condition to be ameliorated.

9. A method according to any one of claims 1-8, wherein the mammal is a human.

10. The method of claim 9, wherein the polypeptide having LIF properties is human LIF.
